(19) United States
(12) Patent Application Publication
(10) Pub. No.: US 2025/0127829 A1
(43) Pub. Date: Apr. 24, 2025

(54) METHOD FOR PREVENTING AND/OR REPAIRING OCULAR CELL DAMAGE BY USING STREPTOCOCCUS THERMOPHILUS STRAIN AND ITS METABOLITES

(71) Applicant: NUTRAREX BIOTECH CO., LTD., Taichung City (TW)

(72) Inventors: Meei-Yn Lin, Taichung City (TW); Pin-Chao Huang, Taichung City (TW); Pei-Cheng Lin, Taichung City (TW); Yi-Wen Chen, Taichung City (TW); Chin-Hsiu Yu, Taichung City (TW); Shao-Yu Lee, Taichung City (TW); Tsung-Han Lu, Taichung City (TW)

(21) Appl. No.: 18/919,470

(22) Filed: Oct. 18, 2024

Related U.S. Application Data
(60) Provisional application No. 63/544,648, filed on Oct. 18, 2023.

(51) Int. Cl. A61K 35/744; A61P 27/02

(52) U.S. Cl. CPC A61K 35/744; A61P 27/02

(57) ABSTRACT

The present disclosure provides a method for preventing and/or repairing ocular cell damage by using a Streptococcus thermophilus iHA318 strain and its metabolites.

### CROSS-REFERENCE TO RELATED APPLICATION

This application claims priority of Provisional application No. 63/544,648, filed on Oct. 18, 2023, the content of which is incorporated herein in its entirety by reference.

### BACKGROUND OF THE INVENTION

### 1. Field of the Invention

The present invention relates to a method for preventing and/or repairing ocular cell damage by using a Streptococcus thermophilus iHA318 strain and its metabolites.

### 2. The Prior Art

Ultraviolet (UV) and hyperosmolarity are common causes of ocular cell damage. Exposure to solar ultraviolet radiation leads to the production of reactive oxygen species (ROS), mitochondrial dysfunction, DNA damage, and can increase cell apoptosis and cause retinal damage. It is also associated with the pathogenesis of a variety of degenerative eye diseases, including macular degeneration (AMD), cataracts, glaucoma and diabetic retinopathy. The corneal epithelium is the outermost layer of the eye surface. Excessive exposure to ultraviolet light can cause corneal damage, increase the risk of bacterial infection, and may also lead to pterygium, conjunctival melanoma, and cataracts.

Hyperosmolarity may cause serious damage to the retina, including causing macular edema, destroying the blood-retinal barrier (BRB), and causing a decrease in the expression of aquaporin AQP4 in retinal pigment epithelial cells. Hyperosmolarity promotes excessive accumulation of water in the retina, aggravates the formation of retinal edema, further damages the retinal structure, and ultimately leads to visual impairment.

In addition, hyperosmolarity of the ocular surface is associated with neurotrophic keratopathy, corneal epithelial disease, Sjogren's disease (SS) and laser assisted in situ keratomileusis (LASIK)-induced neurotrophic epitheliopathy (LNE). Hyperosmolarity causes an imbalance of water and electrolytes inside and outside the corneal epithelial cells, causing the cells to decrease in volume and shrink rapidly, reducing cell survival rate. It also leads to the reduction of conjunctival cells, exacerbating the increase in ocular surface osmolarity, forming a vicious cycle.

Common ocular surface osmoprotectants include hydroxypropylmethylcellulose (HPMC), taurine, betaine (trimethylglycine) and L-carnitine. However, these osmoprotectants have the problem of short effect or poor absorption rate, and their efficacy may be relatively insufficient for the hyperosmolarity of the retina.

From the above, it can be seen that retinal cells and corneal cells are easily damaged by light and hyperosmolarity, and the damage increases with aging, frequency of use of electronic equipment, light pollution, air pollution, pressure or other external factors, exacerbating cell damage such as hyperosmolarity and reduced goblet cell density, increasing the incidence of eye diseases such as retinopathy.

However, the existing technology mainly relies on chemical drugs for treatment, but these methods have a specific effect and must simultaneously protect and repair ocular cell damage. The effect is limited, and they are often accompanied by side effects, such as eye irritation and allergic reactions, etc., which may even aggravate symptoms. Therefore, there is still a strong demand in the market for comprehensive, long-term, safe and side-effect-free treatment options.

In order to solve the above-mentioned problems, those skilled in the art need to develop a safe and effective pharmaceutical composition for the benefit of a large group of people in need thereof.

### SUMMARY OF THE INVENTION

In order to solve the foregoing problems, an objective of the present invention is to provide a method for preventing and/or repairing ocular cell damage, comprising administering to a subject in need thereof a pharmaceutical composition comprising an effective amount of a Streptococcus thermophilus strain, wherein the pharmaceutical composition further comprises a metabolite of the Streptococcus thermophilus strain, and the Streptococcus thermophilus strain is viable.

According to an embodiment of the present invention, the Streptococcus thermophilus strain and the metabolite of the Streptococcus thermophilus strain activate goblet cells on ocular surface.

According to an embodiment of the present invention, the Streptococcus thermophilus strain is deposited in Deutsche Sammlung von Mikroorganismen und Zellkulturen (DSMZ) under an accession number DSM 33978.

According to an embodiment of the present invention, the ocular cell damage is damage to retinal cells and corneal cells caused by ultraviolet (UV) irradiation or hyperosmolarity.

According to an embodiment of the present invention, the damage to retinal cells comprises retinopathy, diabetic retinopathy and macular degeneration.

According to an embodiment of the present invention, the pharmaceutical composition is a medicament, a nutritional supplement, a health food, a food product, a skin health product, an external product, or a combination thereof.

According to an embodiment of the present invention, the medicament comprises a pharmaceutically acceptable carrier.

According to an embodiment of the present invention, the food product comprises an edible material.

According to an embodiment of the present invention, the pharmaceutical composition has a dosage form of powder, granule, solution, gel or paste.

According to an embodiment of the present invention, the pharmaceutical composition is in a dosage form for oral administration.

According to an embodiment of the present invention, the pharmaceutical composition is in a dosage form for parenteral administration.

In summary, the results of the present invention are illustrated by the following examples. The Streptococcus thermophilus iHA318 strain and its metabolites can indeed achieve the effect of preventing and/or repairing ocular cell damage. The present invention provides a safe, comprehensive and effective solution through the Streptococcus thermophilus iHA318 strain and its metabolites. This technology not only protects retinal and corneal cells, but also promotes the activation of ocular surface goblet cells and reduces ocular surface osmolarity, thereby improving the overall health of the eyes. Compared with the prior art, the present invention overcomes the shortcomings of side effects and limited effects, and provides the natural treatment options desired by consumers, with significant market competitive advantages.

The pharmaceutical composition of the present invention comprising a Streptococcus thermophilus strain and its metabolites is used to protect retinal cells (human retinal pigment epithelial cells) and/or corneal cells (human corneal epithelial cells) from UV irradiation and hyperosmolarity injury. It can also reduce the hyperosmolarity of the eyes, activate the conjunctival goblet cells, and maintain overall eye health in an all-round way.

The present invention is mainly used in the art of eye care and treatment, especially for the prevention and treatment of ocular cell damage and osmolarity abnormalities caused by environmental pressure, ultraviolet rays or other external factors. The present invention can also be applied to the development of health foods for eye protection, especially products focusing on improving eye health. In addition, the present invention may also be applied to other medical products related to osmolarity regulation and eye repair formula-related drugs and dressings.

The embodiments of the present invention would be further described below. The following examples are used to illustrate the present invention and are not intended to limit the scope of the present invention. Anyone skilled in the art can make some changes and modifications without departing from the spirit and scope of the present invention. Therefore, the scope of the present invention shall be defined by the appended claims.

### BRIEF DESCRIPTION OF THE DRAWINGS

The following drawings form part of the present specification and are included here to further demonstrate some aspects of the present invention, which can be better understood by reference to one or more of these drawings, in combination with the detailed description of the embodiments presented herein.

FIG. 1 shows the effect of Streptococcus thermophilus iHA318 strain and its metabolites on preventing UV damage to retinal cells. *** P<0.001 indicates a statistically significant difference compared with the UV damage group.

FIG. 2 shows the effect of Streptococcus thermophilus iHA318 strain and its metabolites on preventing retinal cells from hyperosmolarity injury. *** P<0.001 indicates a statistically significant difference compared with the damage group treated with 160 mM NaCl.

FIG. 3 shows the effect of Streptococcus thermophilus iHA318 strain and its metabolites on preventing UV damage to human corneal cell lines. *** P<0.001 indicates a statistically significant difference compared with the UV damage group.

FIG. 4 shows the effect of Streptococcus thermophilus iHA318 strain and its metabolites on preventing hyperosmolarity injury to human corneal cell lines. ** P<0.05 indicates a statistically significant difference compared with the damage group treated with 150 mM NaCl.

FIG. 5 shows that there is no significant change in ocular surface osmolarity after treatment in the placebo group (before treatment: 302.97±2.90 mOsm/L, after treatment: 305.17±3.39 mOsm/L; p=0.312). On the contrary, subjects in the iHA318 group have a significant decrease in ocular surface osmolality after treatment (before treatment: 304.84±2.42 mOsm/L, after treatment: 299.55±1.83 mOsm/L; p=0.043), * P<0.05 compared to day 0.

FIG. 6 shows that the number of goblet cells is sparse due to long-term hyperosmolarity before the patient uses the product. After oral administration of iHA318 strain for 35 days, goblet cells increase from 3.42% before treatment to 92.69% after treatment, which has a significant effect on promoting goblet cell proliferation. *** P<0.001 compared to day 0.

### DETAILED DESCRIPTION OF THE PREFERRED EMBODIMENT

In the following detailed description of the embodiments of the present invention, reference is made to the accompanying drawings, which are shown to illustrate the specific embodiments in which the present disclosure may be practiced. These embodiments are provided to enable those skilled in the art to practice the present disclosure. It is understood that other embodiments may be used and that changes can be made to the embodiments without departing from the scope of the present invention. The following description is therefore not to be considered as limiting the scope of the present invention.

### Definition

As used herein, the data provided represent experimental values that can vary within a range of ±20%, preferably within ±10%, and most preferably within ±5%.

Unless otherwise stated in the context, “a”, “the” and similar terms used in the specification (especially in the following claims) should be understood as including singular and plural forms.

As used herein, the term “preventing” or “prevention” means preventing or delaying symptoms of disease onset when a drug or therapy is used to an individual who does not have symptoms of disease onset but is at high risk of disease onset.

The “metabolites” described herein are substances secreted by a microorganism after being metabolized. More specifically, they can be substances secreted by bacteria into the bacterial culture medium during culture.

The scientific name of Streptococcus thermophilus described herein is “Streptococcus thermophilus”, which is equivalent to “Streptococcus salivarius subsp. thermophilus”.

According to the present invention, the operating procedures and parameter conditions related to bacterial culture fall within the scope of the professional literacy and routine techniques of those skilled in the art.

The Streptococcus thermophilus iHA318 strain of the present invention was deposited in the Deutsche Sammlung von Mikroorganismen und Zellkulturen (DSMZ) on Aug. 17, 2021, under accession number DSM 33978. The Streptococcus thermophilus iHA318 strain will be publicly available upon the granting of the present application.

The pharmaceutical composition provided according to the present invention can be in any suitable form without special limitations. It depends on the desired use and can be in a corresponding suitable dosage form. For example, but not limited thereto, the pharmaceutical composition can be administered to subjects in need via oral or parenteral administration.

According to the present invention, the pharmaceutical composition can be manufactured to a dosage form suitable for parenteral, oral or topical administration, using techniques well known to those skilled in the art, including, but not limited to, injection (e.g., sterile aqueous solution or dispersion), sterile powder, tablet, troche, lozenge, pill, capsule, dispersible powder or granule, solution, suspension, emulsion, syrup, elixir, slurry, external preparation, eye drop, wetting solution, artificial tears, contact lens preservation solution, eye washes, gels and the like.

The pharmaceutical composition according to the present invention may be administered by a parenteral route selected from the group consisting of: intraperitoneal injection, subcutaneous injection, intraepidermal injection, intradermal injection, intramuscular injection, intravenous injection, intralesional injection, sublingual administration, transdermal administration, topical eye administration, and intraocular administration.

The pharmaceutical composition according to the present invention can comprise a pharmaceutically acceptable carrier which is widely used in pharmaceutical manufacturing technology. For example, the pharmaceutically acceptable carrier can comprise one or more reagents selected from the group consisting of solvent, buffer, emulsifier, suspending agent, decomposer, disintegrating agent, dispersing agent, binding agent, excipient, stabilizing agent, chelating agent, diluent, gelling agent, preservative, wetting agent, lubricant, absorption delaying agent, liposome, and the like. In addition, the external product may further comprise an acceptable adjuvant that is widely used in external product manufacturing techniques. For example, the acceptable adjuvant may comprise one or more reagents selected from the group consisting of: solvent, gelling agent, active agent, preservative, antioxidant, screening agent, chelating agent, surfactant, coloring agent, thickening agent, filler, fragrance, and odor absorber. The selection and quantity of these reagents fall within the scope of the professional literacy and routine techniques of those skilled in the art.

According to the present invention, the pharmaceutically acceptable vehicle comprises a solvent selected from the group consisting of water, normal saline, phosphate buffered saline (PBS), aqueous solution containing alcohol, and combinations thereof.

The pharmaceutical composition provided according to the present invention can be administered with different administration frequencies such as once a day, multiple times a day, or once a few days, depending on the individual's needs, age, weight and health status. In the pharmaceutical composition provided according to the present invention, the content ratio in the pharmaceutical composition can be adjusted according to actual application requirements. In addition, the pharmaceutical composition may additionally contain one or more other active ingredients (such as eye medications) as needed, or be used in combination with drugs containing the one or more other active ingredients in order to further strengthen the effect of the pharmaceutical composition or increase the flexibility and deployment degree of the formulation, as long as the other active ingredients have no adverse effect on the benefits of the active ingredients of the present invention (i.e., the Streptococcus thermophilus strain and its metabolites).

Optionally, the medicament or food product provided according to the present invention may additionally contain an appropriate amount of additives, such as flavoring agents, toners, colorants, etc. that can improve the taste and visual perception of the medicament or food product when taking it, and buffers, preservatives, antibacterial agents, antifungal agents, etc. that can improve the stability and storage of the medicament or food product.

The food composition provided by the present invention can be a food product, and is formulated with edible materials to include but not limited to: beverages, fermented foods, bakery products, health foods, nutritional supplements, and dietary supplements.

According to the present invention, the edible material is selected from the group consisting of: water, fluid milk products, milk, concentrated milk; fermented milk such as yogurt, sour milk, frozen yogurt, and lactic acid bacteria-fermented beverages; milk powder; ice cream; cream cheeses; dry cheeses; soybean milk; fermented soybean milk; vegetable-fruit juices; juices; sports drinks; confectionery; jellies; candies; infant formulas; health foods; animal feeds; Chinese herbals; and dietary supplements.

According to the present invention, the food product can be regarded as a food additive, which is added during the preparation of raw materials by conventional methods, or added in the production process of food, and formulated with any edible material into food products for human and non-human animals to eat.

The beverage, fermented food, bakery product, health food, nutritional supplements, and dietary supplements provided according to the present invention can be eaten with different frequencies such as once a day, multiple times a day, or once a few days, depending on the individual's age, weight and health status. The content of the Streptococcus thermophilus strain and its metabolites in the beverage, fermented food, bakery product, health food, nutritional supplement and dietary supplement provided according to the present invention can also be adjusted according to the needs of specific groups, for example, adjusted to be taken every day amount.

For beverages, fermented foods, bakery products, health foods, nutritional supplements and/or dietary supplements provided according to the present invention, the recommended usage amount can be marked on their outer packaging, the usage standards and conditions for specific groups (for example: pregnant woman), or recommendations for co-administration with other foods or medicines, etc., to facilitate users to take them without the guidance of doctors, pharmacists or relevant deacons without safety concerns. In the food product provided according to the present invention, the aspects of the Streptococcus thermophilus strain and its metabolites and related applications are as described above.

The present invention is further illustrated by the following examples. These examples are provided for illustration only and are not intended to limit the scope of the present invention. The scope of the present invention is shown in the appended claims.

The physiological characteristics of the Streptococcus thermophilus iHA318 strain of the present invention are as follows. Growth temperature is 35° C. to 40° C. Growth pH<4.6 after 24 hours of growth in appropriate medium. Oxygen effects are facultative anaerobic. The appearance characteristics of the colonies of the Streptococcus thermophilus iHA318 strain of the present invention are as follows. The edge is complete, the colony is slightly white and larger, and the surface is smooth and raised. The morphological characteristics of the Streptococcus thermophilus iHA318 strain of the present invention are as follows: streptococcal shape, no sporulation, and no mobility. The Gram staining result of the Streptococcus thermophilus iHA318 strain of the present invention is positive.

### Example 1

### Strain Culture

The frozen Streptococcus thermophilus iHA318 strain (hereinafter referred to as the iHA318 strain) (deposited in the Deutsche Sammlung von Mikroorganismen und Zellkulturen (DSMZ) on Aug. 17, 2021, under accession number DSM 33978) is thawed. 1% of the bacteria was inoculated into the MRS liquid medium and cultured in an anaerobic environment at 37° C. for 16-24 hours to activate the iHA318 strain. Subsequently, 1 mL of MRS culture medium containing activated iHA318 strain was added to 100 mL of MRS culture medium, and cultured in an anaerobic environment at 37° C. for 16-24 hours. Next, the MRS medium containing the iHA318 strain was sterilized using an autoclave at 121° C. for 15 minutes to obtain the iHA318 test samples required for subsequent cell experiments.

### Example 2

### Effect of Streptococcus thermophilus iHA318 Strain and its Metabolites on Preventing Retinal Cell Damage from Ultraviolet (UV) Light

Human retinal pigment epithelial cells were cultured in Dulbeco's modified Eagle medium (DMEM)/Ham's F12 containing 10% fetal bovine serum (FBS) and antibiotic mixture (Gibco). The cells were cultured in a 24-well plate at 1×10\hat{ }5 cells/well, and cultured at 37° C. and 5% CO₂ for 24 hours to allow the cells to adhere. The experimental groups were divided into three groups, namely: (1) Control group: only cell culture medium added and no UVA irradiation, (2) Damage group: only cell culture medium added and with UVA irradiation, and (3) iHA318: after appropriately diluting the iHA318 test sample in Example 1, cell culture fluid was added and UVA irradiation treatment was performed. After the cells were attached to the above three groups, they were rinsed with phosphate buffered saline (PBS), and after adding 1 mL, the damage group and iHA318 group were subjected to UVA photodamage treatment. After treatment, the PBS was removed, 1 mL of culture medium was added, followed by incubating for 24 hours at 37° C. and 5% CO₂. The cell culture medium was changed to 250 μL of MTT (3-(4,5-Dimethylthiazol-2-yl)-2,5-diphenyltetrazolium bromide) reagent (Gibco) and incubated at 37°° C. for 2 hours. Subsequently, the MTT reagent was removed, and 500 μL of dimethylsulfoxide (DMSO, Sigma) was added and shaken to evenly dissolve the crystals. An ELISA reader was used to measure the absorbance value at a wavelength of 565 nm and the cell survival rate was calculated.

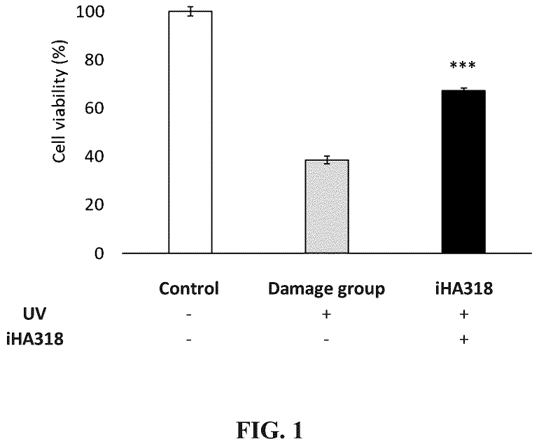

FIG. 1 shows that the survival rate of ARPE-19 cell decreased to 38% after UVA irradiation. After being treated with the iHA318 strain of the present invention, the survival rate of ARPE-19 cells can be significantly increased by 29% compared with the damage group. This result shows that the iHA318 strain of the present invention can reduce cell apoptosis caused by UVA irradiation of retinal cells and effectively prevent retinal cells from being damaged by ultraviolet light.

### Example 3

### Effect of Streptococcus thermophilus iHA318 Strain and its Metabolites on Preventing Retinal Cell Damage Caused by Hyperosmolarity

Human retinal pigment epithelial cells (ARPE-19) were cultured in DMEM/Ham's F12 medium containing 10% fetal bovine serum (FBS) and antibiotic mixture (Gibco). Cells were cultured in 96-well plates at 8×10\hat{ }3 cells/well. After culturing for 24 hours at 37° C. and 5% CO₂ to allow cells to attach, the experimental groups were divided into three groups, namely: control group (general cell culture medium), damage group (medium containing 160 mM NaCl) and iHA318 group (the iHA318 test sample in Example 1 was dissolved in a medium containing 160 mM NaCl at an appropriate concentration). After the cells of the above three groups were attached, 100 μL of the test items of the corresponding groups were added and incubated at 37° C. and 5% CO₂ for 4 hours. The cell culture medium was replaced with 100 μL of MTT (3-(4,5-Dimethylthiazol-2-yl)-2,5-diphenyltetrazolium bromide) reagent (Gibco) and incubated at 37° C. for 2 hours. Subsequently, the MTT reagent was removed, and 100 μL of dimethylsulfoxide (DMSO, Sigma) was added and shaken to evenly dissolve the crystals. An ELISA reader was used to measure the absorbance value at a wavelength of 565 nm and calculate the cell survival rate.

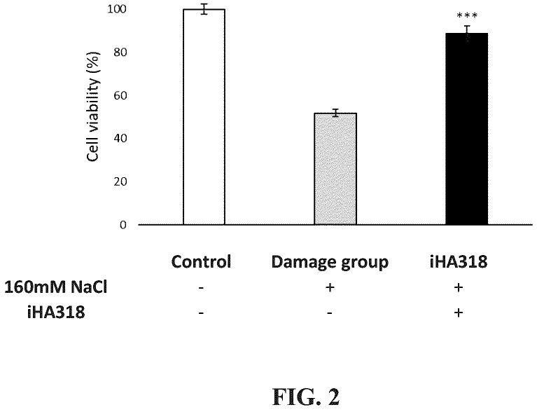

FIG. 2 shows that using high salt to simulate a hyperosmolarity environment causes damage to ARPE-19 cells and reduces the survival rate of ARPE-19 to 52%. After being treated with iHA318 of the present invention, the survival rate of ARPE-19 cells can be significantly increased by 37% compared with the damage group. This result shows that iHA318 of the present invention can reduce the apoptosis of retinal cells caused by hyperosmolarity and effectively prevent retinal cells from being damaged by hyperosmolarity.

### Example 4

### Effect of Streptococcus thermophilus iHA318 Strain and its Metabolites on Preventing UV Damage to Human Corneal Cell Lines

Human corneal cell line (HCE-T) was cultured in DMEM/Ham's F1 2medium containing 5% fetal bovine serum (FBS), 5 μg/ml insulin, 10 ng/ml human epidermal growth factor (human EGF) and 0.5% DMSO. The cells were cultured in a 6-cm culture dish at 7×10\hat{ }4 cells/well and cultured at 37° C. and 5% CO₂ for 24 hours.

After the cells were attached, the experimental groups were divided into three groups, namely: (1) Control group: only cell culture medium added and no UVA irradiation, (2) Damage group: only cell culture medium added and with UVA irradiation, and (3) iHA318: after appropriately diluting the iHA318 test sample in Example 1, cell culture fluid was added and UVA irradiation treatment was performed. The above three groups were rinsed once with PBS after cell attachment, and 4 mL of the test items of the corresponding groups were added. Incubation was performed at 37° C., 5% CO₂ for 24 hours, rinsed with PBS and 4 mL was added. The above three groups were placed in a UV nucleic acid fixation instrument for 10J UVA treatment, and PBS was removed after UVA treatment. 4 mL of culture medium containing sample was added again and incubated at 37° C. and 5% CO₂ for 24 hours. The cell culture medium was replaced with 4 mL of medium-diluted MTT reagent (Gibco) and incubated at 37° C. for 2 hours. Subsequently, the MTT (3-(4,5-Dimethylthiazol-2-yl)-2,5-diphenyltetrazolium bromide) reagent was removed, and 2 mL of dimethylthiazol (DMSO, Sigma) was added to uniformly dissolve the crystals. An ELISA reader was used to measure the absorbance value at a wavelength of 565 nm and calculate the cell survival rate.

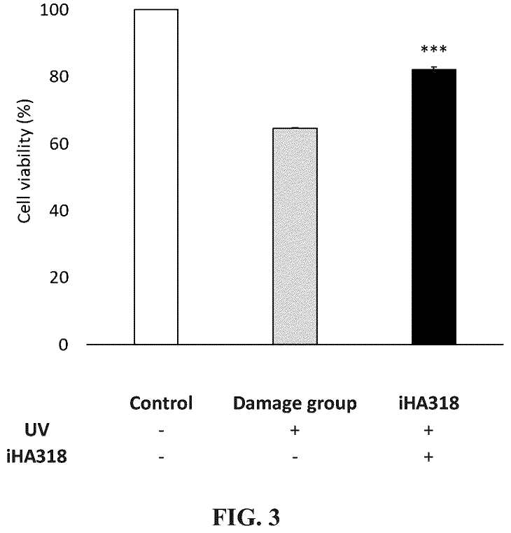

FIG. 3 shows that the cell survival rate of HCE-T decreases to 65% after UVA irradiation. After being treated with iHA318 of the present invention, the survival rate of HCE-T cells can be significantly increased by 18% compared with the damage group. This result shows that iHA318 of the present invention can reduce the apoptosis of corneal cells caused by UVA irradiation, and effectively prevent corneal cells from being damaged by ultraviolet light.

### Example 5

### Effect of Streptococcus thermophilus iHA318 Strain and its Metabolites on Preventing Hyperosmolarity Damage to Human Corneal Cell Lines

Human corneal cell line (HCE-T) was cultured in DMEM/Ham's F12 medium containing 5% fetal bovine serum (FBS), 5 μg/ml insulin, 10 ng/ml human epidermal growth factor (human EGF) and 0.5% DMSO. The cells were cultured in a 96-well plate at 7.0×10\hat{ }3 cells/well and cultured at 37° C. and 5% CO₂ for 24 hours. After the cells were attached, the experimental groups were divided into three groups, namely: control group (general cell culture medium), damage group (medium containing 150 mM NaCl) and iHA318 group (the iHA318 test sample in Example 1was dissolved in a medium containing 150 mM NaCl at an appropriate concentration).

After cells were attached, rinsed once with PBS and 100 μL of the test items of the corresponding group were added. After culturing for 24 hours at 37° C. and 5% CO₂, washed and medium containing 150 mM NaCl or general medium was added depending on the group. After 24 hours, analysis was performed. The culture medium was replaced with 100 μL of appropriately diluted AlamarBlueTM reagent and incubated at 37° C. for 4 hours. Then the absorbance values at wavelengths of 570 nm and 600 nm were measured in an ELISA reader, and the cell survival rate was calculated.

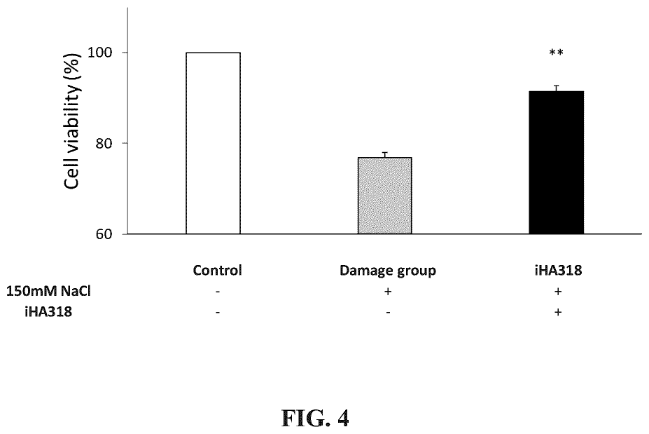

FIG. 4 shows that using high salt to simulate a hyperosmolarity environment causes HCE-T cell damage and reduces the HCE-T survival rate to 76.9%. After being treated with iHA318 of the present invention, the survival rate of HCE-T cells can be significantly increased by 14.5% compared with the damage group. This result shows that iHA318 of the present invention can reduce the apoptosis of corneal cells caused by hyperosmolarity and effectively prevent corneal cells from being damaged by hyperosmolarity.

### Example

### Human Trial of Streptococcus thermophilus iHA318 Strain Repairing Ocular Cell Damage

In one embodiment of the present invention, in order to test the human efficacy of the Streptococcus thermophilus iHA318 strain of the present invention on repairing ocular cell damage, 68 subjects aged between 20 and 65 years old were recruited to conduct a randomized, double-blind and placebo-controlled trial. Placebo group and iHA318 group were set. The iHA318 group took 5.0×10\hat{ }9 of the Streptococcus thermophilus iHA318 strain of the present invention every day for 35 days, and relevant tests were conducted before and after taking it.

Osmolarity analysis was performed by taking 50 nl of tears from subjects before and after using iHA318 strain or placebo. Osmolarity was measured using a TearLab™ osmolarity meter (TearLab™ Corp., San Diego, CA, USA).

Changes in conjunctival goblet cells were analyzed using “imprint cytology staining method”. The nitrocellulose filter paper was attached to the subject's conjunctiva and then removed, and periodic acid-Schiff (PAS) staining or NLRP3 immunofluorescence staining was performed. PAS staining was used to assess goblet cell number. The filter paper was fixed with alcohol, oxidized with periodic acid, rinsed with water twice, and then Schiff's reagent was added and reacted for 15 minutes. After washing with water twice, hematoxylin (Sigma-Aldrich, PAS staining kit, Cat#3952016) was added for staining, followed by rinsing twice with water for mounting and photomicrography. Goblet cells were recognized under a light microscope by their characteristic red or pink morphology. Counting was performed on each filter paper in at least ten fields and the average was calculated.

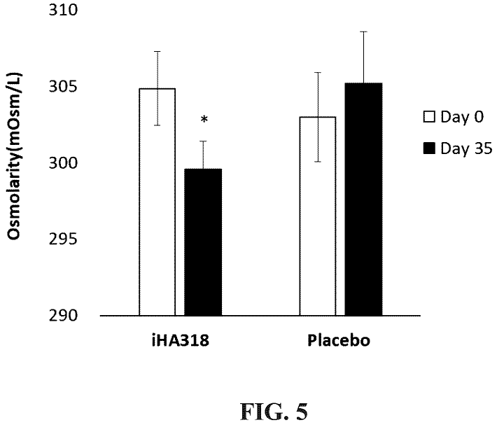

FIG. 5 shows that there is no significant change in ocular surface osmolarity after treatment in the placebo group (before treatment: 302.97±2.90 mOsm/L, after treatment: 305.17±3.39 mOsm/L; p=0.312). On the contrary, subjects in the iHA318 group have a significant decrease in ocular surface osmolality after treatment (before treatment: 304.84±2.42 mOsm/L, after treatment: 299.55±1.83 mOsm/L; p=0.043), showing that iHA318 strain can effectively reduce osmolarity and reduce the damage to eyes caused by hyperosmolarity.

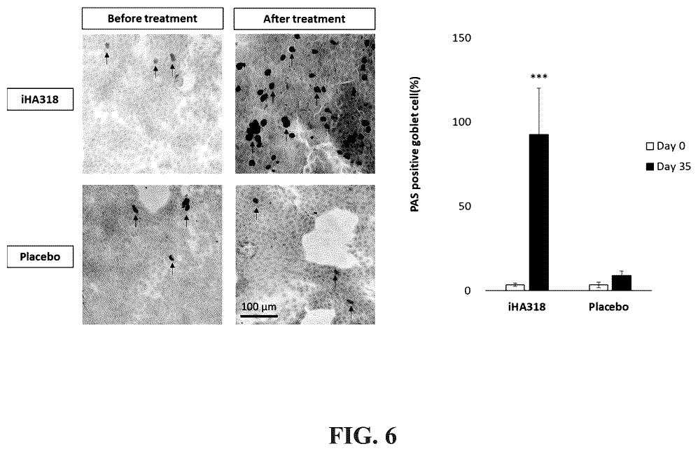

Conjunctival goblet cells are critical to ocular surface health and visual function. FIG. 6 shows that the number of goblet cells is sparse due to long-term hyperosmolarity before the patient uses the product. After oral administration of iHA318 strain for 35 days, goblet cells increase from 3.42% before treatment to 92.69% after treatment, which has a significant effect on promoting goblet cell proliferation. In contrast, no significant improvement was observed in the placebo group, showing that iHA318 strain can promote goblet cell proliferation and repair damage to maintain eye health.

In summary, the results of the present invention are illustrated by the above examples. The Streptococcus thermophilus iHA318 strain and its metabolites can indeed achieve the effect of preventing and/or repairing ocular cell damage. The present invention provides a safe, comprehensive and effective solution through the Streptococcus thermophilus iHA318 strain and its metabolites. This technology not only protects retinal and corneal cells, but also promotes the activation of ocular surface goblet cells and reduces ocular surface osmolarity, thereby improving the overall health of the eyes. Compared with the prior art, the present invention overcomes the shortcomings of side effects and limited effects, and provides the natural treatment options desired by consumers, with significant market competitive advantages.

The pharmaceutical composition of the present invention comprising a Streptococcus thermophilus strain and its metabolites is used to protect retinal cells (human retinal pigment epithelial cells) and/or corneal cells (human corneal epithelial cells) from UV irradiation and hyperosmolarity injury. It can also reduce the hyperosmolarity of the eyes, activate the conjunctival goblet cells, and maintain overall eye health in an all-round way.

The present invention is mainly used in the art of eye care and treatment, especially for the prevention and treatment of ocular cell damage and osmolarity abnormalities caused by environmental pressure, ultraviolet rays or other external factors. The present invention can also be applied to the development of health foods for eye protection, especially products focusing on improving eye health. In addition, the present invention may also be applied to other medical products related to osmolarity regulation and eye repair formula-related drugs and dressings.

Although the present invention has been described with reference to the preferred embodiments, it will be apparent to those skilled in the art that a variety of modifications and changes in form and detail may be made without departing from the scope of the present invention defined by the appended claims.

What is claimed is:

1. A method for preventing and/or repairing ocular cell damage, comprising administering to a subject in need thereof a pharmaceutical composition comprising an effective amount of a Streptococcus thermophilus strain, wherein the pharmaceutical composition further comprises a metabolite of the Streptococcus thermophilus strain, and the Streptococcus thermophilus strain is viable.

2. The method according to claim 1, wherein the Streptococcus thermophilus strain and the metabolite of the Streptococcus thermophilus strain activate goblet cells on ocular surface.

3. The method according to claim 1, wherein the Streptococcus thermophilus strain is deposited in Deutsche Sammlung von Mikroorganismen und Zellkulturen (DSMZ) under an accession number DSM 33978.

4. The method according to claim 1, wherein the ocular cell damage is damage to retinal cells and corneal cells caused by ultraviolet (UV) irradiation or hyperosmolarity.

5. The method according to claim 4, wherein the damage to retinal cells comprises retinopathy, diabetic retinopathy and macular degeneration.

6. The method according to claim 1, wherein the pharmaceutical composition is a medicament, a nutritional supplement, a health food, a food product, a skin health product, an external product, or a combination thereof.

7. The method according to claim 6, wherein the medicament comprises a pharmaceutically acceptable carrier.

8. The method according to claim 6, wherein the food product comprises an edible material.

9. The method according to claim 1, wherein the pharmaceutical composition has a dosage form of powder, granule, solution, gel or paste.

10. The method according to claim 1, wherein the pharmaceutical composition is in a dosage form for oral administration.

11. The method according to claim 1, wherein the pharmaceutical composition is in a dosage form for parenteral administration.
